(19) United States
(12) Patent Application Publication
(10) Pub. No.: US 2008/0177109 A1
(43) Pub. Date: Jul. 24, 2008

(54) Novel Process for Preparation of Bicalutamide

(75) Inventors: Venkatasubramanian Radhakrishnan Tarur, Maharashtra (IN); Suresh Mahadev Kadam, Maharashtra (IN); Anil Purushottam Joshi, Maharashtra (IN); Sachin Baban Gavhane, Maharashtra (IN)

Correspondence Address: PHARMACEUTICAL PATENT ATTORNEYS, LLC, 55 MADISON AVENUE, 4TH FLOOR, MORRISTOWN, NJ, 07960-7397, US

(73) Assignee: USV LIMITED, Mumbai (IN)

(21) Appl. No.: 11/574,886

(22) PCT Filed: May 10, 2005
(86) PCT No.: PCT/IN05/00152

§ 371 Date: Mar. 8, 2007

(30) Foreign Application Priority Data
Mar. 29, 2005 (IN) 363/MUM/2005

(51) Int. Cl. C07C 315/02

(52) U.S. Cl. 564/162

(57) ABSTRACT

The present invention discloses a process for the synthesis of N-[4-Cyano-3-(trifluoromethyl)phenyl]-3-[(4-fluorophenyl)sulphonyl]-2-hydroxy-2-methyl propanamide (Form I). The invention discloses a reagent for oxidation of N-[4-Cyano-3-(trifluoromethyl)phenyl]-3-[(4-fluorophenyl)thio]-2-hydroxy-2-methyl propanamide to N-[4-Cyano-3-(trifluoromethyl)phenyl]-3-[(4-fluorophenyl)sulphonyl]-2-hydroxy-2-methyl propanamide. More particularly, the invention discloses a method of purification of N-[4-Cyano-3-(trifluoromethyl)phenyl]-3-[(4-fluorophenyl)sulphonyl]-2-hydroxy-2-methyl propanamide in a mixture of methylethyl ketone and hexane giving form (I). This form (I) is useful as an active pharmaceutical and has antiandrogenic activity.

### RELATED APPLICATION

This application claims priority from Indian patent application No. 363/MUM/2005, filed on 29/03/2005.

### TECHNICAL FIELD

The present invention relates to an improved process for the preparation of N-[4-Cyano-3-(trifluoromethyl)phenyl]-3-[(4-fluorophenyl)sulphonyl]-2-hydroxy-2-methyl propanamide (Form I).

### BACKGROUND AND PRIOR ART

Bicalutamide is the generic name for the compound N-[4-Cyano-3-(trifluoromethyl)phenyl]-3-[(4-fluorophenyl)sulphonyl]-2-hydroxy-2-methyl propanamide and is represented by the formula (I).

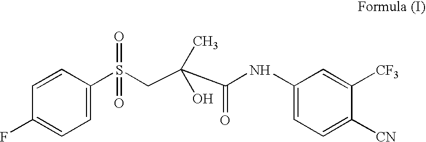

Bicalutamide and related acylanilides have been disclosed in EP 100172 and corresponding U.S. Pat. No. 4,636,505 as pharmaceutically active compounds possessing antiandrogenic activity useful in the treatment of prostrate cancer. Bicalutamide the pharmaceutical product is approved worldwide under the brand name Casodex (Astra Zeneca).

Bicalutamide has been prepared by reacting 3-Trifluoro-methyl-4-cyanoaniline of Formula (IV) with methacryloyl chloride of Formula (III) followed by epoxidation of the resultant N-(3-trifluoromethyl-4-cyanophenyl)methacrylamide of Formula (V). The epoxide ring is opened with 4-fluorothiophenol and subsequent conversion to sulfone resulted in Bicalutamide of Formula (I) as reported in U.S. Pat. No. 4,636,505 issued to ICI and Tucker et. al. J. med. Chem, 31, 9-954-959 (1988).

Bicalutamide is a non-steroidal pharmaceutically active agent possessing antiandrogenic properties, generally used in treatment of prostate cancer i.e. for androgen deprivation treatment, although other androgen dependent conditions may also be treated. Bicalutamide is commercially available in a pharmaceutical composition as a racemate under the brand name Casodex (Astra-Zeneca). The stereoisomer of Bicalutamide has been proposed in U.S. Pat. No. 5,985,868 as being more beneficial than the racemate. Method of preparation for Bicalutamide is disclosed in U.S. Pat. No. 4,636,505, WO0224638, U.S. Pat. No. 6,479,692, WO02100339, US20030073742, US 20030045742.

Several methods are known in the art for making N-[4-Cyano-3-(trifluoromethyl)phenyl]-3-[(4-fluorophenyl)sulphonyl]-2-hydroxy-2-methyl propanamide i.e Bicalutamide from its precursor (VII) i.e N-[4-cyano-3-(trimethyl)phenyl]-3-[(4-fluorophenyl)thio]-2-hydroxy-2-methyl propanamide (VII) using different oxidising agents. The oxidation is accomplished by using various peracids.

WO0224638 discloses the above oxidation using 30% H₂O₂ in presence of trifluoro acetic anhydride in methylene dichloride (CH₂Cl₂) at 25° C. to 30° C.

WO0353920 claims oxidation process of N-[4-cyano-3-(trimethyl)phenyl]-3-[(4-fluorophenyl)thio]-2-hydroxy-2-methyl propanamide (VII) using H₂O₂/Sodium tungstate/Phenyl phosphoric acid/TBAB/Ethyl acetate.

EP0100172, WO0134563, WO02100339 and Tucker et al in J. Med. Chem. 954-959 disclose the oxidation of N-[4-cyano-3-(trimethyl)phenyl]-3-[(4-fluorophenyl)thio]-2-hydroxy-2-methyl propanamide(VII) using m-chloro perbenzoic acid (m-CPBA) in chlorinated solvent, which requires longer hours for reaction. Thus the process disclosed in the prior art involves the use of costly reagents and chlorinated solvents.

Chlorinated solvents are known to be harmful to humans with a suggested possibility of being carcinogenic and also produce dioxin during disposal. Further solvents like CH₂Cl₂ involves higher cost of disposal due to corrosion during incineration.

Further the chemical risk reduction policy, “Green Chemistry” is gaining attention and industrially feasible environment friendly chemical reactions (avoiding, as far as possible, the use of harmful chemicals and developing reactions which do not as far as possible discharge these) are becoming an essential feature in research. The above-mentioned reaction using CH₂Cl₂ as organic solvent is from this point of view not suited for the method of preparation of the desirable Bicalutamide.

Besides being an expensive reagent, MCPBA is a highly explosive material and therefore not desirable industrially.

Synthesis of Bicalutamide without the use of m-CPBA is published in WO0100608. According to this a solution of H₂O₂ is used as oxidizing agent and the compound is oxidized in acetic or formic acid and is considered as an excellent industrial method environmentally and economically for conversion of Formula (VII) to Formula (I). However in this method, both polar and non-polar impurities are formed which is not reduced during purification. Further this method also has a step involving use of halogenated organic solvent (e.g. 1,1,1-trichloroethane) for the synthesis of (VI) and so cannot be considered environmentally friendly.

Bicalutamide synthesis without the use of MCPBA as oxidizing agent is reported in WO0224638. According to this method, H₂O₂ is added to compound of Formula (VII) and the mixture after cooling to −55° C., anhydrous trifluoroacetic acid (TFA) is added to the mixture to get Bicalutamide. But in this method the use of explosive TFA as reagent and the need for cooling during the addition of TFA makes the method uneconomical. Further anhydrous TFA is corrosive and hygroscopic.

WO03053920 has claimed the process of oxidation using H₂O₂/Sodium tungstate/Phenyl phosphoric acid/TBAB/Ethyl acetate in good yield. Here the large excess of H₂O₂ (3 to 6 equivalents) per mole equivalent of the compound of Formula (VII) and use of large quantity of Sodium tungstate or phenyl phosphoric acid, 0.5 to 5% quantity of compound. Further removal of Sodium tungstate and Phenyl phosphoric acid from the reaction mixture is tedious.

So, the present invention is aimed to provide an improved process for the synthesis of Bicalutamide (Form I) in high purity and high yield, which involves the use of inexpensive, non-hazardous and easily available oxidizing agent.

### OBJECTIVES OF THE INVENTION

An object of the present invention is to provide an improved, industrially viable and cost effective process for the preparation of Bicalutamide.

Another object of the present invention is to provide an improved process to obtain Bicalutamide in high yield and substantial purity.

Yet another aspect of the present invention is to provide a novel process for making Bicalutamide Form I.

### SUMMARY

The present invention discloses a process for the synthesis of N-[4-Cyano-3-(trifluoromethyl)phenyl]-3-[(4-fluorophenyl)sulphonyl]-2-hydroxy-2-methyl propanamide (Form I). The invention discloses a novel reagent i.e. sodium perborate in presence of acetic acid for oxidation of N-[4-Cyano-3-(trifluoromethyl)phenyl]-3-[(4-fluorophenyl)thio]-2-hydroxy-2-methyl propanamide to N-[4-Cyano-3-(trifluoromethyl)phenyl]-3-[(4-fluorophenyl)sulphonyl]-2-hydroxy-2-methyl propanamide. The invention also relates to a novel method of purification of N-[4-Cyano-3-(trifluoromethyl)phenyl]-3-[(4-fluorophenyl)sulphonyl]-2-hydroxy-2-methyl propanamide in a mixture of methylethyl ketone and hexane giving form (I) in substantially pure form.

### DETAILED DESCRIPTION

The present invention describes a process for preparing N-[4-Cyano-3-(trifluoromethyl)phenyl]-3-[(4-fluorophenyl)sulphonyl]-2-hydroxy-2-methyl propanamide Form I of formula (I), known as Bicalutamide. However the inventive process can also be used to prepare any compound within the scope of formula (I) including those disclosed in patents WO0224638, WO0100608, WO03053920 and U.S. Pat. No. 4,636,505 all of which are incorporated by reference in their entirety herein.

The invention also provides the purification of N-[4-Cyano-3-(trifluoromethyl)phenyl]-3-[(4-fluorophenyl)sulphonyl]-2-hydroxy-2-methyl propanamide by precipitating it from a solution of N-[4-Cyano-3-(trifluoromethyl)phenyl]-3-[(4-fluorophenyl)sulphonyl]-2-hydroxy-2-methyl propanamide in a ketone to obtain substantially pure bicalutamide (Form I).

The solvent used for dissolution of bicalutamide is a ketone; preferably methylethyl ketone. The cosolvent for precipitation is preferably hydrocarbon and most preferably hexane. The precipitation is preferably carried out at a temperature between 30° C. to 50° C., more preferably between 35° C. to 40° C. and most preferably at about 40° C. The cosolvent (i.e. hydrocarbon) is added over a period of about 1 to 3 hrs, more preferably about 1 to 2 hrs and most preferably for about 1 hrs 30 minutes. When the precipitation is complete, the product is filtered and is washed with hexane.

The crystalline solid (referred to as ‘Form I’) exhibits an X-ray powder diffraction pattern as shown in FIG. 1. The 2θ and ‘d’ values matched the values reported in WO2004029021 for the product obtained by a process as disclosed in U.S. Pat. No. 4,636,505 that teaches use of Ethyl acetate/Petroleum ether for making Form I.

The N-[4-Cyano-3-(trifluoromethyl)phenyl]-3-[(4-fluorophenyl)sulphonyl]-2-hydroxy-2-methyl propanamide of formula (I) prepared by oxidation of N-[4-Cyano-3-(trifluoromethyl)phenyl]-3-[(4-fluorophenyl)thio]-2-hydroxy-2-methyl propanamide of Formula (VII) with sodium perborate tetrahydrate. A preferred solvent is acetic acid. Stoichiometric ratio of the substrate to sodium perborate tetrahydrate is preferably 1:10, more preferably 1:5 and most preferably 1:2.2 molar ratios may be used to minimize formation of impurity. The reaction is preferably carried out at temperature between 40° C. to 60° C., more preferably between 40° C. to 50° C. Sodium perborate tetrahydrate is preferably added in about 1-2 hrs. The reaction requires preferably 6 to 10 hrs at about 45° C. for completion. The reaction is complete when unreacted N-[4-Cyano-3-(trifluoromethyl)phenyl]-3-[(4-fluorophenyl)sulphonyl]-2-hydroxy-2-methyl propanamide in the reaction mixture is 1% by HPLC. The reaction mixture is cooled to 20 to 25° C. and isolated by filtration. The obtained N-[4-Cyano-3-(trifluoromethyl)phenyl]-3-[(4-fluorophenyl)sulphonyl]-2-hydroxy-2-methyl propanamide of Formula (I) has high purity i.e. at least 99% as measured by HPLC. The product of Formula I was also obtained in good yield i.e. about 90% with respect to the N-[4-Cyano-(3-trifluoromethyl)phenyl]-3-[(4-fluorophenyl)thio]-2-hydroxy-2-methyl propanamide. The synthesis of Bicalutamide(I) is as shown in Scheme I:

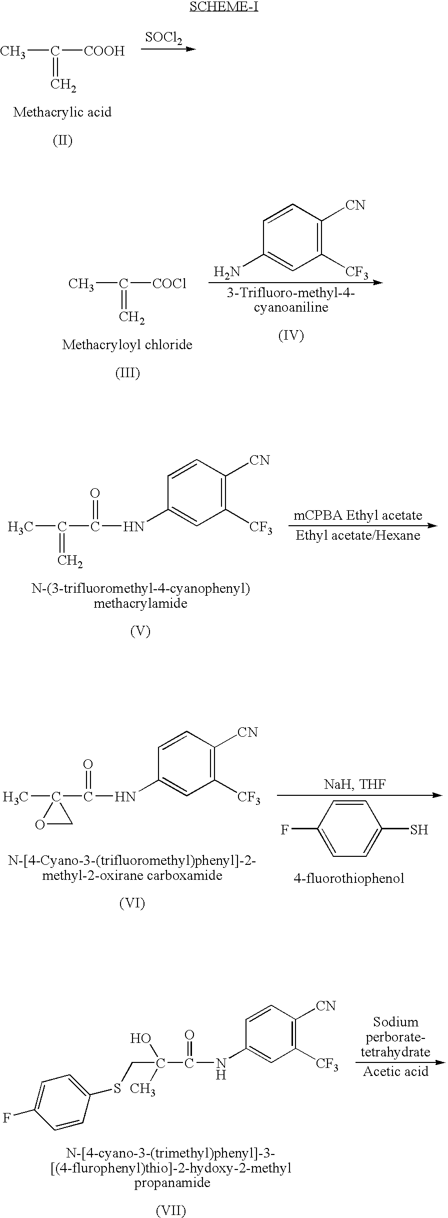

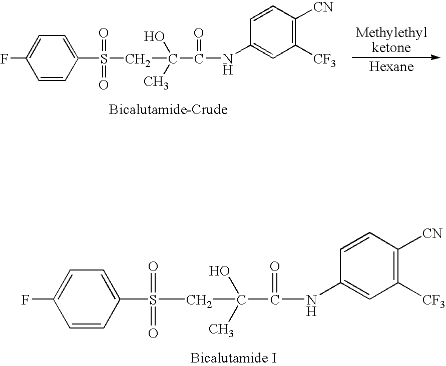

### BRIEF DESCRIPTION OF THE DRAWINGS

FIG. 1 show the XRPD of conventional Bicalutamide form I.

TABLE 1

| 2-Theta | d value angstrom |
|---|---|
| 6.060, 9.337, | 14.57232, 9.46462, |
| 11.656, 12.122, | 7.58571, 7.29545, |
| 13.754, 14.144, | 6.43339, 6.25679, |
| 14.390, 14.857, | 6.15616, 5.95795, |
| 15.628, 16.399, | 5.66590, 5.40121, |
| 16.854, 17.180, | 5.25611, 5.15724, |
| 18.273, 18.662, | 4.85114, 4.75089, |
| 18.960, 19.550, | 4.67685, 4.53710, |
| 22.145, 22.493, | 4.01090, 3.94962, |
| 23.230, 23.652, | 3.82596, 3.75861, |
| 24.586, 24.814, | 3.61790, 3.58517, |
| 27.306, 27.803, | 3.26345, 3.20622, |
| 28.609, 29.495 | 3.11772, 3.02597, |
| 30.065, 30.706, | 2.96991, 2.90940, |
| 31.332, 31.780, | 2.85266, 2.81348, |
| 32.068, 32.619, | 2.78888, 2.74299, |
| 33.774, 34.119, | 2.65177, 2.65572, |
| 34.590, 36.786, | 2.59105, 2.44124, |
| 37.009, 37.380, | 2.42708, 2.40385, |
| 39.810, 43.476. | 2.26252, 2.07987 |

The present invention is illustrated in further detail with reference to the following non-limiting examples.

### EXAMPLE-1

### Preparation of N-[4-Cyano-3-(trifluoromethyl)phenyl]-3-[(4-fluorophenyl)sulphonyl]-2-hydroxy-2-methyl propanamide

200 ml (210.6 gm) of glacial acetic acid and 25 gm (0.062 mole) of N-[4-Cyano-3-(trifluoromethyl)phenyl]-3-[(4-fluorophenyl)thio]-2-hydroxy-2-methyl propanamide (99%) of Formula (VII) are charged into a 500 ml reactor at 25 to 30° C. Subsequently 22 gm (0.127 mole) of sodium perborate tetrahydrate was charged at about 25 to 30° C. in a period of 1 hr. The reaction temperature was maintained at this temperature for about 1 hr. The suspension was heated to about 45° C. and this temperature was maintained for 8 hrs at which time a sample was withdrawn. If the starting thio compound was less than 1.0%, then the suspension was cooled to 20 to 25° C. and stirred further for 4 hrs. The product was filtered from the reaction mixture at this temperature and the solid was washed with 250 ml of water, and then with 100 ml of n-Hexane. The solid isolated was dried at 60° C. for 8 hrs.

24.3 gm of Bicalutamide (crude) of Formula (I) was obtained with a yield of 90% and purity of about 99% by HPLC.

### EXAMPLE-2

### Preparation of N-[4-Cyano-3-(trifluoromethyl)phenyl]-3-[(4-fluorophenyl)sulphonyl]-2-hydroxy-2-methyl propanamide (Form I)

88 ml methylethyl ketone and 22 gm crude Bicalutamide as obtained in example 1 are charged into a 500 ml reactor. The mixture was heated to about 80° C. to obtain solution. Subsequently 0.2 gm charcoal was added and stirred for further 0.5 hr and then charcoal was filtered off and the cake was washed with 10 ml of hot methylethyl ketone. To the clear colourless solution 88 ml Hexane was added at 50° C. in 1 hr. Then the reaction mixture was cooled to 25 to 30° C. slowly and further cooled to 10 to 15° C. in 1 hr, where white crystalline product precipitate out. The product was filtered and washed with 22 ml mixture of methylethyl ketone and hexane. The solid was dried at 60° C. for 8 hrs. 19.8 gm of solid was obtained with yield of 90.0% and purity greater than 99.8% by HPLC.

We claim:

1) A process of preparation of Bicalutamide Form I comprising oxidation of N-[4-Cyano-3-(trifluoromethyl)phenyl]-3-[(4-fluorophenyl)thio]-2-hydroxy-2-methyl propanamide of Formula (VII) with sodium perborate trihydrate in presence of a solvent.

2) The process as claimed in claim 1 wherein preferred solvent is acetic acid.

3) The process as claimed in claim 1, wherein the oxidation is carried out at about 40° C.-60° C.

4) The process as claimed in claim 1, wherein the reaction is complete in about 6 to 12 hrs.

5) The process as claimed in claim 1, wherein the product is isolated at about 20° C.-25° C.

6) A process of obtaining a substantially pure Bicalutamide comprising precipitating Bicalutamide (form I) from a solution containing Bicalutamide.

7) The process as claimed in claim 6 wherein said precipitation is carried out by contacting said bicalutamide solution in methylethyl ketone with a contra solvent.

8) The process as claimed in claim 7 wherein said contra solvent is hexane.

9) The process as claimed in claim 6 to 8, wherein said precipitation occurs at a temperature of about 30° C. to 50° C.

10) The Bicalutamide as claimed in claims 6 to 9, having an X-ray diffraction peak of Form I.

11) The process as claimed in claims 6 to 10 wherein the bicalutamide obtained is about 99% pure.

12) The process as claimed in claims 6 to 10 wherein the purity of bicalutamide obtained is greater than 99.8%.

13) A process for the preparation of Bicalutamide and its purification as substantially described herein with reference to the foregoing examples 1-2.
